(19) United States
(12) Patent Application Publication
(10) Pub. No.: US 2026/0117322 A1
(43) Pub. Date: Apr. 30, 2026

(54) MOLECULAR MARKER FOR IDENTIFYING VITIS VINIFERA AND INTERSPECIFIC HYDRID GRAPES AND APPLICATION THEREOF

(71) Applicant: INSTITUTE OF BOTANY,THE CHINESE ACADEMY OF SCIENCES, Beijing (CN)

(72) Inventors: Zhenchang LIANG, Beijing (CN); Kangyi DING, Beijing (CN); Yi WANG, Beijing (CN); Peige FAN, Beijing (CN); Yangfu KUANG, Beijing (CN); Wei DUAN, Beijing (CN); Chong REN, Beijing (CN); Yang LI, Beijing (CN)

(21) Appl. No.: 19/338,028

(22) Filed: Sep. 24, 2025

(30) Foreign Application Priority Data
Oct. 29, 2024 (CN) 202411518151.6

(51) Int. Cl. C12Q 1/6895

(52) U.S. Cl. CPC C12Q 1/6895; C12Q 2600/156; C12Q 2600/16

(57) ABSTRACT

A molecular marker for identifying Vitis vinifera and its interspecific hybrid grapes and an application thereof are provided. Through whole-genome resequencing data analysis, a significant InDel locus IH1 is identified, which is located at 7,224,357 base pairs (bp) on chromosome 13 of grapes. This locus shows genotypic differences between Vitis vinifera and its interspecific hybrids. Through the differences in the number and distribution of electrophoretic bands, Vitis vinifera and its interspecific hybrids may be distinguished. Based on the characteristics of this molecular marker, it may be used for screening of interspecific hybrid progeny lines in the early stage of breeding and elimination of selfed progeny. At the same time, it may also conduct screening of the genetic background of seedlings.

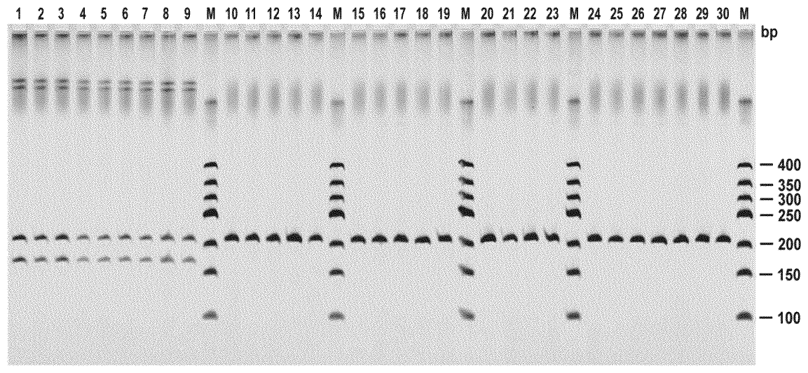

### CROSS-REFERENCE TO RELATED APPLICATIONS

This application claims priority to Chinese Patent Application No. 202411518151.6, filed on Oct. 29, 2024, the contents of which are hereby incorporated by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: SequenceListing.xml
Creation date: Sep. 22, 2025
Byte size: 5,044

### TECHNICAL FIELD

The present disclosure relates to the field of biological breeding technology, and in particular to a molecular marker for identifying Vitis vinifera and its interspecific hybrid grapes and an application thereof.

### BACKGROUND

Grapes are one of the world's important economic crops with extremely high economic value. In recent years, with the advancement of agricultural technology and the diversification of consumer demand, China's grape planting area and yield have steadily increased, making the grape industry one of the most important fruit industries in the country. Especially in the table grape and wine grape industries, China's market demand continues to expand, which imposes higher requirements on the diversity and quality of grape varieties.

In grape breeding, interspecific hybridization is a widely used genetic improvement method that may introduce excellent genetic resources not present in cultivated species, thereby enhancing grape genetic diversity. Through interspecific hybridization, desirable traits such as disease resistance, cold tolerance, and drought resistance may be transferred from wild species to cultivated varieties. Common hybrid combinations include crosses between Vitis vinifera and Amerimay grape species (e.g., Vitis labrusca, Vitis riparia), which are mainly used to improve disease resistance and cold tolerance; as well as crosses between Vitis vinifera and wild grape species (e.g., Vitis amurensis, Vitis aestivalis), aiming at enhancing cold tolerance and stress resistance.

The phenotypes of interspecific hybrid progeny are usually similar to those of their parents, making it difficult to accurately identify hybrid authenticity by using traditional morphological, agronomic, or physiological-biochemical indexes, especially under complex hybridization backgrounds. Screening using these traditional methods is not only affected by environmental conditions but also inefficient and costly. To overcome these limitations, modern molecular biology techniques have been widely adopted to improve identification accuracy and efficiency. The specific genomic differences exhibited by interspecific hybrid progeny make molecular markers an ideal tool. Since interspecific hybrid progeny originate from the combination of genes from different species, they may exhibit heterozygous states, whereas Vitis vinifera remains homozygous at specific loci. These genotypic differences provide an effective basis for distinguishing Vitis vinifera from its hybrid progeny. Such markers may also be used for genetic background identification of seedlings, reducing varietal admixture of seedlings and rootstocks, and errors in the use of seedlings.

With the development of molecular biology, molecular marker technology has become a key tool for germplasm resource identification. Through genome-level analysis, molecular markers may rapidly identify hybrids in the early stages of breeding, significantly shortening the breeding cycle. They may also be used for rapid genetic background analysis of seedlings before planting to ensure their accuracy and reduce economic losses caused by seedling issues. However, current molecular marker technologies for identifying grape interspecific hybrids still face challenges such as insufficient marker specificity, complex technical operations, and high detection costs. Therefore, developing an efficient and specific molecular marker identification method will provide important technical support for rapid screening of grape interspecific hybrids and seedling detection, further advancing the development of grape breeding.

### SUMMARY

The objectives of the disclosure are to provide a molecular marker for identifying Vitis vinifera and its interspecific hybrid grapes and an application thereof, addressing the aforementioned issues in existing technologies. This molecular marker may accurately distinguish Vitis vinifera from its interspecific hybrid grapes, improving breeding efficiency and accuracy while reducing breeding costs.

To achieve the above objectives, the disclosure provides the following schemes:
the present disclosure provides a molecular marker for identifying Vitis vinifera and its interspecific hybrid grapes, where a nucleotide sequence of the molecular marker is as shown in SEQ ID NO: 1, and a 34 base-pair (bp) deletion polymorphism is present starting from a 49th position of the molecular marker.

Optionally, if a 34 bp deletion starting from the 49th position of the molecular marker is present and a genotype is heterozygous, a grape is an interspecific hybrid; if the 34 bp deletion starting from the 49th position is absent and the genotype is homozygous, the grape is Vitis vinifera; where a sequence of the 34 bp deletion is as shown in SEQ ID NO: 4.

The present disclosure also provides a primer pair for identifying Vitis vinifera and its interspecific hybrid grapes, where the primer pair is used to amplify the molecular marker with the nucleotide sequence as shown in SEQ ID NO: 1, and nucleotide sequences of the primer pair are as shown in SEQ ID NO: 2-3.

The present disclosure also provides a kit including the primer pair.

The present disclosure also provides a method for identifying Vitis vinifera and its interspecific hybrid grapes, including following steps:
using genomic deoxyribonucleic acid (DNA) of a test grape sample as a template, performing Polymerase chain reaction (PCR) amplification with the primer pair according to claim 3, and judging whether the test grape sample is Vitis vinifera or an interspecific hybrid grape based on the number and distribution differences of bands at a marker locus in an amplification product; where the marker locus is located at positions 49-82 of a nucleotide sequence as shown in SEQ ID NO: 1;
Optionally, a PCR amplification reaction system includes: 25 microliters (μL) of 2×Rapid Taq Master Mix, 2 μL each of an upstream sequence and a downstream sequence, 0.1-1 microgram (g) of DNA template, and ddH2O supplementation to a total volume of 50 μL.

Optionally, the PCR amplification reaction program is: 94 degrees Celsius (° C.) denaturation for 5 minutes (min); 94° C. denaturation for 30 seconds (s), 59° C. annealing for 30 s with annealing temperature decreasing by 0.5° C. per cycle, 72° C. extension for 30 s, for a total of 5 cycles; 94° C. denaturation for 30 s, 56° C. annealing for 30 s, 72° C. extension for 30 s, for a total of 25 cycles; followed by a final extension at 72° C. for 5 min; and storage at 4° C.

Optionally, in a judgment method, if double bands appear at the marker locus, the sample is judged as the interspecific hybrid grape; and if a single band appears at the marker locus, the sample is judged as the Vitis vinifera grape.

The present disclosure also provides an application of the molecular marker, the primer pair, or the kit in identifying Vitis vinifera and its interspecific hybrid grapes.

The present disclosure also provides an application of the molecular marker, the primer pair, or the kit in assisting grape breeding.

The disclosure has the following technical effects.

Through in-depth analysis of whole-genome resequencing data, the disclosure selects a marker locus with significant genotypic differences, namely the InDel locus at 7,224,357 bp on chromosome 13 of grape. The 34 bp deletion at this locus causes interspecific hybrid grapes to exhibit double bands in the electrophoretogram, while Vitis vinifera exhibits a single band. This stable genetic difference ensures that the disclosure has high accuracy in identifying interspecific hybrid grapes, effectively avoiding misclassification.

The molecular marker system of the disclosure provides accurate genetic background classification in the early stages of grape breeding. This precise genetic information support enables breeders to rapidly screen individuals that meet breeding objectives and eliminate individuals that have not been effectively hybridized, thereby shortening the breeding cycle and improving breeding efficiency. At the same time, the molecular marker may also be used for seedling identification, determining their genetic background and providing molecular-level evidence for seedling evaluation and identification, and ensuring seedling accuracy.

Compared with traditional methods, the single-locus marker system of the disclosure significantly simplifies the detection process, and reduces reliance on multiple marker loci. This simplification lowers reagent consumption and operational steps, making large-scale screening and classification more economical and feasible, thereby significantly reducing overall germplasm identification costs.

The molecular marker technology of the disclosure not only improves the accuracy of identifying grape interspecific hybrids, but also provides reliable scientific evidence for the registration and protection of new grape varieties. By accurately distinguishing interspecific hybrids from Vitis vinifera, the technology effectively prevents variety confusion, safeguarding the legality and uniqueness of varieties. Furthermore, the disclosure supports the registration, certification, and intellectual property protection of grape seedlings, further enhancing their market value and legal rights and interests of varieties.

### BRIEF DESCRIPTION OF THE DRAWING

To more clearly illustrate the embodiments of the disclosure or the technical schemes in the prior art, the following will briefly describe the drawing required for the embodiments. Obviously, the drawing in the following description is only some embodiments of the disclosure. For those skilled in the art, other drawings may be obtained without creative effort based on these drawings.

The FIGURE is the electrophoretic typing results of the molecular marker IH1 in interspecific hybrid and Vitis vinifera grapes; where M represents the 500-base pair (bp) deoxyribonucleic acid (DNA) Marker, and lanes 1-30 correspond to the grape varieties Beixiang, Beifeng, Beizi, Beiquan, Xinbeichun, Beichun, Beihong, Beixin, Beimei, Sangiovese, Syrah, Ugni Blanc, Aligoté, Riesling, Domfelder, Chardonnay, Pinot Gris, Pinot Blanc, Pinot Noir, Petit Manseng, Italian Riesling, Petit Verdot, Malbec, Sauvignon Blanc, Grenache, Marselan, Cabernet Sauvignon, Cabernet Gernischet, Cabernet Franc, and Merlot, respectively.

### DETAILED DESCRIPTION OF THE EMBODIMENTS

Various exemplary embodiments of the disclosure are now described in detail. This detailed description should not be construed as limiting the disclosure but rather as providing a more detailed description of certain aspects, features, and embodiments of the disclosure.

It should be understood that the terminology used herein is for the purpose of describing particular embodiments only and is not intended to limit the disclosure. Additionally, for numerical ranges recited herein, it should be understood that each intermediate value between the upper and lower limits of the range is also specifically disclosed. Any stated value or intermediate value within a stated range, and any smaller range formed by any other stated or intermediate value within the stated range, is also included in the disclosure. The upper and lower limits of these smaller ranges may be independently included or excluded from the range.

Unless otherwise defined, all technical and scientific terms used herein have the same meaning as commonly understood by one of ordinary skill in the art to which the disclosure belongs. Although only optional methods and materials are described herein, any methods and materials similar or equivalent to those described may also be used in the practice or testing of the disclosure. All publications mentioned herein are incorporated by reference to disclose and describe the methods and/or materials related to the publications. In case of conflict with any incorporated publication, the content of this specification shall prevail.

Various modifications and changes may be made to the specific embodiments of the disclosure described herein without departing from the scope or spirit of the disclosure, as will be apparent to those skilled in the art. Other embodiments derived from the description of the disclosure will also be apparent to those skilled in the art. The description and examples herein are illustrative only.

The terms “comprising,” “including,” “having,” “containing,” etc., used herein are open-ended terms, meaning including but not limited to.

### Embodiment 1 Screening and Identification of Molecular Marker IH1 for Vitis Vinifera and its Hybrid Grapes

The disclosure selects representative Vitis vinifera grapes (Cabernet Sauvignon, Cabernet Franc, Pinot Noir, etc.) and their interspecific hybrids (Beihong, Beimei, etc.) for whole-genome resequencing. After standardizing the sequencing data, a significant InDel locus (locus information is shown in Table 1) is identified at 7,224,357 base-pair (bp) on chromosome 13 of grapes, with the reference genome being V. vinifera PN40024 (12×). At this locus, significant differences exist between interspecific hybrids and Vitis vinifera grapes. The wild parent of the interspecific hybrid has a 34 bp deletion, causing the hybrid progeny to carry different alleles and exhibit a heterozygous state, whereas Vitis vinifera grapes do not have this deletion and remain homozygous. Based on this locus, the molecular marker IH1 (nucleotide sequence is as shown in SEQ ID NO: 1) is designed to specifically amplify the target fragment. Vitis vinifera grapes and their interspecific hybrids may be intuitively distinguished through the number and distribution differences of electrophoretic bands.

The sequence of molecular marker IH1 is:

CCCCAGGCTATTTGTGAAGAAGATGAAGAAGATGACAAAAATGAC
AATAAGATCAAGGCAAATTCTTAGTATTCATCGACTCAGAAATCT
TATCTCAAGCAAAAGTATCAAATGAATATTAGATCAGGCATTACA
CTGGTTAATTTAGAGGTTACAATTGTATGTAACAAATAAAGCTGA
TTTCTGTATCCACCCAAA.
The underlined portion in the above
sequence represents the 34 bp deletion sequence.

TABLE 1
Sequence information of the InDel
locus corresponding to marker IH1

| | | | Reference | |
|---|---|---|---|---|
| | Chromo- | | sequence | |
| Marker | some | Position | (5′-3′) | Allele |
| VvISH1 | 13 | 7224357 | AAGATCAAG | A |
| | | | GCAAATTCT | |
| | | | TAGTATTCA | |
| | | | TCGACTC | |
| | | | (SEQ ID | |
| | | | NO: 4) | |

This embodiment mainly includes the following steps:

### (1) Genomic Data Analysis and Variant Information Acquisition

Representative Vitis vinifera grapes and their interspecific hybrids are selected for whole-genome resequencing. The sequencing data are standardized, and variant loci information with significant genetic differences are extracted.

### (2) Marker Region Selection and Primer Design

A locus with genotypic differences is selected, and a marker region is screened within approximately 150 bp upstream and downstream of this locus. Specific primers are designed for classification and identification. The selected marker region contains relatively stable genetic variability, and its flanking sequences are consistent. Such dominant markers may effectively distinguish Vitis vinifera grapes from their interspecific hybrids.

### (3) Sample Preparation and Deoxyribonucleic Acid (DNA) Extraction

Young grape tissues (such as leaves, young stems, roots) or, in special cases, mature tissues are selected. Genomic DNA is extracted using the modified CTAB method to obtain high-quality DNA samples, so as to ensure the accuracy and reliability of subsequent analyses.

### (4) Polymerase Chain Reaction (PCR) Amplification

Touchdown-PCR is used for amplification with a reaction system volume of 50 microliters (μL). This method improves amplification specificity by gradually reducing the annealing temperature, thereby enhancing the detection capability of the target fragment.

TABLE 2
Reaction system

| Reagent | Volume |
|---|---|
| 2x Rapid Taq Master Mix | 25 μL |
| Primer 1 (10 micromolars (μM)) | 2 μL |
| Primer 2 (10 μM) | 2 μL |
| Template DNA | 0.1-1 microgram (μg) |
| ddH₂O | to 50 μL |

The reaction program is: 94 degrees Celsius (° C.) denaturation for 5 minutes (min); 94° C. denaturation for 30 seconds (s), 59° C. annealing for 30 s with annealing temperature decreasing by 0.5° C. per cycle, 72° C. extension for 30 s, for a total of 5 cycles; 94° C. denaturation for 30 s, 56° C. annealing for 30 s, 72° C. extension for 30 s, for a total of 25 cycles; followed by a final extension at 72° C. for 5 min; and storage at 4° C.

Primer 1:
(SEQ ID NO: 2)
5′-CCCCAGGCTATTTGTGAAGA-3′;
Primer 2:
(SEQ ID NO: 3)
5′-TTTGGGTGGATACAGAAATCAG-3′.

### (5) Electrophoresis and Result Verification

Polyacrylamide gel electrophoresis is performed, and silver staining method is used for visualization. The electrophoretic results show that interspecific hybrids exhibit double bands, while Vitis vinifera grapes exhibit a single band. This is due to genotypic differences at the marker locus. Interspecific hybrids have a 34 bp deletion, resulting in different alleles and showing a heterozygous state, whereas Vitis vinifera grapes do not have this variation and remain homozygous. Through the number and distribution differences of bands, Vitis vinifera grapes from their interspecific hybrids may be distinguished.

### Embodiment 2 Application of Molecular Marker IH1 in Identifying Vitis Vinifera and its Hybrid Grapes

This embodiment selects 9 interspecific hybrids (Beixiang, Beifeng, Beizi, Beiquan, Xinbeichun, Beichun, Beihong, Beixin, Beimei) independently bred in the laboratory and 21 Vitis vinifera wine grapes (Sangiovese, Syrah, Ugni Blanc, Aligoté, Riesling, Domfelder, Chardonnay, Pinot Gris, Pinot Blanc, Pinot Noir, Petit Manseng, Italian Riesling, Petit Verdot, Malbec, Sauvignon Blanc, Grenache, Marselan, Cabernet Sauvignon, Cabernet Gernischet, Cabernet Franc, Merlot) for validation of the typing effect of marker IH1 as shown in Table 3 are selected to verify the typing effect of marker IH1.

### 1. Sampling and DNA Extraction

Young leaves and stems of hybrid seedlings are collected, and DNA is extracted using the modified CTAB plant genomic DNA rapid extraction kit.

TABLE 3
Names of hybrid seedling germplasm resources

| Serial | | | |
|---|---|---|---|
| number | Name | Germplasm category | Species name (Latin name) |
| 1 | Beixiang | V. vinifera × V. thunbergii | V. vinifera × V. thunbergii cv. |
| | | hybrid | Beixiang |
| 2 | Beifeng | V. vinifera × V. thunbergii | V. vinifera × V. thunbergii cv. |
| | | hybrid | Beifeng |
| 3 | Beizi | V. vinifera × V. thunbergii | V. vinifera × V. thunbergii cv. |
| | | hybrid | Beizi |
| 4 | Beiquan | V. vinifera × V. amurensis | V. vinifera × V. amurensis cv. |
| | | hybrid | Beiquan |
| 5 | Xinbeichun | V. vinifera × V. amurensis | V. vinifera × V. amurensis cv. |
| | | hybrid | Xinbeichun |
| 6 | Beichun | V. vinifera × V. amurensis | V. vinifera × V. amurensis cv. |
| | | hybrid | Beichun |
| 7 | Beihong | V. vinifera × V. amurensis | V. vinifera × V. amurensis cv. |
| | | hybrid | Beihong |
| 8 | Beixin | V. vinifera × V. amurensis | V. vinifera × V. amurensis cv. |
| | | hybrid | Beixin |
| 9 | Beigong | V. vinifera × V. amurensis | V. vinifera × V. amurensis cv. |
| | | hybrid | Beigong |
| 10 | Sangiovese | V. vinifera | V. vinifera cv. Sangiovese |
| 11 | Syrah | V. vinifera | V. vinifera cv. Syrah |
| 12 | Ugni Blanc | V. vinifera | V. vinifera cv. Ugni Blanc |
| 13 | Aligote | V. vinifera | V. vinifera cv. Aligote |
| 14 | Riesling | V. vinifera | V. vinifera cv. Riesling |
| 15 | Dornfelder | V. vinifera | V. vinifera cv. Dornfelder |
| 16 | Chardonnay | V. vinifera | V. vinifera cv. Chardonnay |
| 17 | Pinot Gris | V. vinifera | V. vinifera cv. Pinot Gris |
| 18 | Pinot Blanc | V. vinifera | V. vinifera cv. Pinot Blanc |
| 19 | Pinot Noir | V. vinifera | V. vinifera cv. Pinot Noir |
| 20 | Petit Manseng | V. vinifera | V. vinifera cv. Petit Manseng |
| 21 | Italian Riesling | V. vinifera | V. vinifera cv. Italian Riesling |
| 22 | Petit Verdot | V. vinifera | V. vinifera cv. Petit Verdot |
| 23 | Malbec | V. vinifera | V. vinifera cv. Malbec |
| 24 | Sauvignon | V. vinifera | V. vinifera cv. Sauvignon Blanc |
| | Blanc | | |
| 25 | Grenache | V. vinifera | V. vinifera cv. Grenache |
| 26 | Marselan | V. vinifera | V. vinifera cv. Marselan |
| 27 | Cabernet | V. vinifera | V. vinifera cv. Cabernet |
| | Sauvignon | | Sauvignon |
| 28 | Cabernet | V. vinifera | V. vinifera cv. Cabernet |
| | Gernischet | | Gernischet |
| 29 | Cabernet Franc | V. vinifera | V. vinifera cv. Cabernet Franc |
| 30 | Merlot | V. vinifera | V. vinifera cv. Merlot |
| M | 400 bp DNA | / | / |
| | Marker | | |

### 2. DNA Quality Detection

The purity and concentration of DNA samples are detected using an ultra-micro ultraviolet-visible spectrophotometer and 1 percent (%) agarose gel electrophoresis. DNA purity and concentration are measured with the ultra-micro ultraviolet spectrophotometer, when D_{260/280} is between 1.8 and 2.0, they are considered high-purity. Meanwhile, 1% agarose gel electrophoresis is used to detect the purity and concentration of DNA samples. Clear and uniform bands indicate high quality of DNA extraction; if tailing occurs, it indicates DNA degradation, and re-extraction is required. Finally, combining these two methods, DNA concentrations are diluted to approximately 200 nanograms per microliter (ng/μL) and stored at 4° C.; for long-term storage, it may be placed at −20° C.

### 3. PCR Amplification

### 3.1 Primer Design

Specific primers are designed based on the InDel locus at 7,224,357 bp on chromosome 13 of grapes, as shown below:

TABLE 4
Information on specific amplification primers
for molecular marker IH1

| | | Annealing | Product |
|---|---|---|---|
| Primer | 5′→3′ Sequence | Temperature | Length |
| IH1-F | CCCCAGGCTATTTGTGAAGA | 60.07 | 198 bp |
| | (SEQ ID NO: 2) | | |
| IH1-R | TTTGGGTGGATACAGAAATC | 58.95 | |
| | AG (SEQ ID NO: 3) | | |

### 3.2 PCR Reaction System

All operations are performed on ice. The total volume of PCR amplification reaction system is 50 μL, which is the same as shown in Table 2 of Embodiment 1.

### 3.3 PCR Parameters

To improve the specificity of amplification products, Touchdown-PCR is used to amplify the target fragment. The specific program is: 94° C. denaturation for 5 min; 94° C. denaturation for 30 s, 59° C. annealing for 30 s (with the annealing temperature decreasing by 0.5° C. per cycle), 72° C. extension for 30 s, for 5 cycles; 94° C. denaturation for 30 s, 56° C. annealing for 30 s, 72° C. extension for 30 s, for 25 cycles; final extension at 72° C. for 5 min; and storage at 4° C.

### 4. Polyacrylamide Gel Electrophoresis

### (1) Preparation of Electrophoresis Gel

A 12% polyacrylamide gel is used in this standard, with gel area of 306×95 mm². The preparation process is as follows: the long and short glass plates are thoroughly rinsed with detergent and tap water, then rinsed with distilled water 1-2 times, and then air-dried. The surfaces of the plates are sprayed with 95% ethanol, and after the ethanol volatilizes and it is confirmed that there are no impurities. The long gel plate is placed horizontally on a flat stand of the experimental platform, the short gel plate is then placed on top of it, and both sides of the gel plates are clamped with clips. After assembly, the bottom is sealed with 1.5% agarose. 70 milliliters (mL) of 12% non-denaturing gel is prepared. In a clean conical flask, 24.5 mL of distilled water, 28 mL of 30% polyacrylamide (29:1), 25 mL SDS-PAGE separation gel buffer, 700 μL of 10% ammonium persulfate, and finally 28 μL of TEMED are added sequentially to a clean conical flask. After mixing well, the gel solution is poured slowly and horizontally into the gel chamber formed by the two glass plates to avoid bubble formation. If bubbles are generated during pouring, the glass plates are gently tapped to remove them. Finally, the comb is inserted, and the gel is allowed to polymerize for 1-2 hours (h).

### (2) Electrophoresis Parameters

A vertical electrophoresis tank is used with an electrophoresis voltage of 220 Volt (V) and a current that varies with voltage. Electrophoresis is performed for 1 h.

### (3) Staining

Silver staining is used to visualize the polyacrylamide gel electrophoresis results, and images are taken for documentation. The specific steps are:
(a) fixation: taking down the gel and placing it in a tray containing 250 mL of 10% glacial acetic acid, ensuring that the solution covers the gel surface by about 5 millimeters (mm), and shaking in parallel for 15 min.
(b) Washing: pouring off the acetic acid solution and rinsing the gel with distilled water three times, 2 min each time.
(c) Staining: pouring off the distilled water and adding 250 mL of 0.2% AgNO₃ solution into the tray, then shaking in parallel for 30 min.
(d) Washing: pouring off the staining solution, adding distilled water once for washing, and shaking for about 10 s.
(e) Color development: adding 250 mL of 3% NaOH solution and 1.25 mL of formaldehyde into the tray, and shaking in parallel for about 10 min until color development.
(f) Fixation: pouring off the development solution, adding 250 mL of 10% glacial acetic acid, and shaking for 2 min to determine the reaction.
(g) Preservation: pouring off the fixing solution, and adding distilled water to preserve the gel.

### 5. Result Analysis

As shown in the FIGURE, the sample numbering order in the FIGURE corresponds to that in Table 3. The electrophoretic results show that all interspecific hybrids exhibit double bands, with amplification products appearing at 198 bp and 164 bp, while Vitis vinifera grapes exhibit a single band, with amplification product appearing at 198 bp. This is due to genotypic differences at the marker locus: interspecific hybrids have a 34 bp deletion, resulting in different alleles and showing a heterozygous state, whereas Vitis vinifera grapes lack this variation and show a homozygous state. This demonstrates that the molecular marker of the disclosure may effectively distinguish Vitis vinifera grapes from their interspecific hybrids based on the number and distribution differences of bands.

The above-described embodiments are merely illustrative of the optional embodiments of the disclosure and are not intended to limit the scope of the disclosure. Without departing from the spirit of the disclosure, various modifications and improvements made by those skilled in the art shall fall within the scope of the disclosure as defined by the appended claims.

What is claimed is:

1. A molecular marker for identifying Vitis vinifera and interspecific hybrid grapes thereof, wherein a nucleotide sequence of the molecular marker is as shown in SEQ ID NO: 1, and a 34 base-pair (bp) deletion polymorphism is present starting from a 49th position of the molecular marker.

2. The molecular marker according to claim 1, wherein when a 34 bp deletion starting from the 49th position of the molecular marker is present and a genotype is heterozygous, a grape is an interspecific hybrid; when the 34 bp deletion starting from the 49th position is absent and the genotype is homozygous and the grape is Vitis vinifera; wherein a sequence of the 34 bp deletion is as shown in SEQ ID NO: 4.

3. A primer pair for identifying Vitis vinifera and interspecific hybrid grapes thereof, wherein the primer pair is used to amplify a molecular marker with a nucleotide sequence as shown in SEQ ID NO: 1, and nucleotide sequences of the primer pair are as shown in SEQ ID NO: 2-3.

4. A method for identifying Vitis vinifera and interspecific hybrid grapes thereof, comprising following steps:
using genomic deoxyribonucleic acid (DNA) of a test grape sample as a template, performing Polymerase chain reaction (PCR) amplification with the primer pair according to claim 3, and judging whether the test grape sample is a Vitis vinifera grape or an interspecific hybrid grape based on number and distribution differences of bands at a marker locus in an amplification product; wherein the marker locus is located at positions 49-82 of the nucleotide sequence as shown in SEQ ID NO: 1; and
wherein in a judgment method, when double bands appear at the marker locus, the test grape sample is judged as the interspecific hybrid grape; and when a single band appears at the marker locus, the test grape sample is judged as the Vitis vinifera grape.

5. The method according to claim 4, wherein a PCR amplification reaction system comprises: 25 microliters (μL) of 2×Rapid Taq Master Mix, 2 μL each of an upstream sequence and a downstream sequence, 0.1-1 microgram (g) of DNA template, and ddH₂O supplementation to a total volume of 50 μL.

6. The method according to claim 4, wherein a PCR amplification reaction program is: 94 degrees Celsius (° C.) denaturation for 5 minutes (min); 94° C. denaturation for 30 seconds (s), 59° C. annealing for 30 s with annealing temperature decreasing by 0.5° C. per cycle, 72° C. extension for 30 s, for a total of 5 cycles; 94° C. denaturation for 30 s, 56° C. annealing for 30 s, 72° C. extension for 30 s, for a total of 25 cycles; followed by a final extension at 72° C. for 5 min; and storage at 4° C.


SEQUENCE LISTING (4 sequences)

SEQ ID NO: 1 - DNA, 198 bp, synthetic construct
  source 1..198: mol_type = other DNA
ccccaggctatttgtgaagaagatgaagaagatgacaaaaatgacaataagatcaaggcaaattcttagtattcatcgactcagaaatcttatctcaagcaaaagtatcaaatgaatattagatcaggcattacactggttaatttagaggttacaattgtatgtaacaaataaagctgatttctgtatccacccaaa

SEQ ID NO: 2 - DNA, 20 bp, synthetic construct
  source 1..20: mol_type = other DNA
ccccaggctatttgtgaaga

SEQ ID NO: 3 - DNA, 22 bp, synthetic construct
  source 1..22: mol_type = other DNA
tttgggtggatacagaaatcag

SEQ ID NO: 4 - DNA, 34 bp, synthetic construct
  source 1..34: mol_type = other DNA
aagatcaaggcaaattcttagtattcatcgactc